(19) United States
(12) Patent Application Publication
(10) Pub. No.: US 2012/0296305 A1
(43) Pub. Date: Nov. 22, 2012

(54) Male Light Incontinence Hygienic Article

(76) Inventors: Maria F. Barraza Khaled, Buenos Aires (AR); Lazlo Belmont, Lima (PE); Veronica Cowan-Ros, Southlake, TX (US); Juan D. Garces, Medellin (CO); Esteban Meléndez Vega, Heredia (CR); Eduardo A. Poppe, Lima (PE); Patricia E. Riquelme, Buenos Aires (AR); Percy Silva, Lima (PE)

(21) Appl. No.: 13/434,462

(22) Filed: Mar. 29, 2012

Related U.S. Application Data
(60) Provisional application No. 61/477,926, filed on Apr. 21, 2011.

(51) Int. Cl. A61F 13/45

(52) U.S. Cl. 604/385.16

(57) ABSTRACT

A hygienic article for personal wear by a male, the hygienic article includes an elongate, liquid permeable, and at least uni-directionally stretchable fabric sleeve having a first end, and a longitudinally opposite second end. A central passage extends longitudinally between the first and second ends of the sleeve, the first end of the sleeve being open to the central passage to permit a wearer's penis to extend through the open first end into the central passage of the sleeve. The second end of the sleeve is permanently affixed to an absorbent cap, either bonded to an end of the sleeve or contained within a closed end of the sleeve, which absorbent cap controls leakage of liquid at the tip of the user's penis.

### PRIORITY

This non-provisional application claims priority of Provisional Application No. 61/477,926, filed on Apr. 21, 2011. The entirety of Application No. 61/477,926 is incorporated herein by reference.

### FIELD OF INVENTION

This invention relates generally to hygienic articles for personal wear by males to take in and retain body fluids away from the wearer's skin, and more particularly to such hygienic articles which are intended to be worn on a male's penis to take in and retain residual fluids, and yet which provide comfort, ease of use, and reduce or avoid potential for skin irritation.

### BACKGROUND

The skin of a male's penis is particularly sensitive, and may be exposed to various bodily fluids such as perspiration, urine and semen that can lead to poor skin health and hygiene. For example, residual urine is sometimes left on the skin of the penis following urination, or may leak from the penis following urination or due to light incontinence, a condition common in aging males. Such incidents of residual urination are even more prevalent with atypical physiological conditions such as hypospadias (where the urethra is open on the underside of the shaft of the penis), epispadias (where the urethra is open on the upper side of the shaft of the penis), and following various types of abdominal surgeries (such as for example, following prostate surgery), where it is not uncommon for male patients to thereafter suffer from chronic light incontinence.

One possible solution for guarding the skin of the penis against such residual fluid loss is to wear abdominal coverage incontinence garments, which are absorbent garments (and more typically disposable absorbent garments) that resemble and are worn in the manner of conventional undergarments, such as diapers or shield-like products. Such products are typically worn either around a user's torso or waist, or are inserted inside a user's undergarments to lie either against the user's penis, or in a cup-like fashion, around the user's scrotum and penis (entire genital region). However, such garments are intended more for taking in and retaining a more substantial volume of liquid, such as where an entire urination event, or insult, occurs.

Such traditional incontinence garments may be bulky and therefore non-discrete, often presenting emotional and/or psychological obstacles to the wearing of such products. Moreover, only certain portions of the wearer's penis would sufficiently contact the incontinence garment, meaning that residual fluids would still remain on other and potentially larger portions of the skin including the abdomen, buttocks, and groin. Female hygienic articles such as pantyliners or pads may also adequately function to some extent as inserts but such articles are distinctively associated with women and would also present emotional and psychological obstacles for a male wearer.

In the development of products which cater to these particular male incontinence needs, numerous product designs have been created which surround either the tip of the male penis (glans penis), the shaft of the male penis, the tip and the shaft, or alternatively, the entire genital area including the scrotum area. Such devices may be characterized as either 1) urinary occlusion devices, designed specifically for either providing a seal at the end of the penis, thereby physically blocking the flow of urine from the penis, such as by creating a dam or alternatively, exerting pressure on the penis to prevent leakage through the urethra, or 2) urinary capture devices. For example, urinary incontinence devices designed for sealing are described in U.S. Pat. No. 6,131,575 to Lenker et al. and US2006/0276764 to Warne. Such devices provide some protection for light leakage, but may often sacrifice comfort given the sealing or fastening means employed with such structures. Examples of tubular cover device designs purportedly used to absorb light leakage may be found in U.S. Pat. No. 5,643,235 to Figuerido, U.S. Pat. No. 6,949,090 to Leers, US2011/0054428 to Hill, and U.S. Pat. No. 4,601,716 to Smith. Such tubular structures are often accompanied with restrictive or awkward fastening systems such as belts, tapes or mechanical hook and loop fasteners, or have liquid impermeable portions along the penile shaft region so as to prevent garment soiling. While such devices are somewhat successful in preventing leakage, they are often irritating, as they maintain moisture close to the skin in many areas (as a result of their use of moisture impermeable barrier sheets), and can also be uncomfortable from a thermal perspective. Cap-like incontinence devices designed to fit about the outer tip of a male's penis may be easily removed by the wearer, but can also be easily dislodged inadvertently, during normal movements from daily activities. Further, cap-like incontinence devices may not sufficiently reduce the release of odors associated with residual urine or urine leakage.

An example of a full male genital covering may be found in U.S. Pat. No. 5,036,839 to Weiss. However, as previously described, such devices often utilize uncomfortable attachment mechanisms for their operation. Additional designs for male incontinence devices include those which have separately attachable absorbent portions, such as that described in WO2007/078370 to Bester et al., and which include a separate penile shaft sleeve to allow for free urination by the user. Such designs include multiple separable components.

While numerous devices exist to address male light incontinence, there is a further need for a discrete and comfortable hygienic article for wearing on a male's penis to take-in and retain residual fluids released by the wearer, while allowing for healthy skin ventilation, minimizing moisture contact with adjacent body surfaces and release of odor, and while also allowing for the wear by a diverse group of users, and provide for normal movement of the penis during day-to-day activities.

### SUMMARY

In one aspect, the present invention is directed to a hygienic article for personal wear by a male. The hygienic article generally comprises an elongate stretchable or elastic, and breathable, desirably liquid permeable, fabric sleeve portion having a first end, a longitudinally opposite second end, and a central passage extending longitudinally between the first and second ends of the sleeve. The first end of the elongate sleeve is open to permit the wearer's penis to extend through the open first end into the central passage of the sleeve. The first end of the sleeve is nearer to the base of the wearer's penis than the second end of the sleeve. The sleeve is constructed of at least a uni-directional stretchable or elastic breathable sheet, but desirably a multi-directional stretchable or elastic breathable sheet, which is desirably liquid permeable and is permanently affixed to an absorbent cap portion which is liquid impermeable.

In another aspect, the present invention is directed to a hygienic article for personal wear by a male, generally comprising an elongate elastic and liquid permeable fabric sleeve having a first end, a longitudinally opposite second end, and a central passage extending longitudinally between the first and second ends of the sleeve. The first end of the sleeve is open to permit the wearer's penis to extend through the open first end into the central passage of the sleeve. The first end of the sleeve is nearer to the base of the wearer's penis than the second end of the sleeve. The second end of the sleeve also is permanently affixed to an absorbent cap portion which is liquid impermeable and which includes a secondary barrier, in the form of an elastic collar or hydrophobic polymer for prevention of wicking of fluid to/along the sleeve portion.

In another aspect, the present invention is directed to a hygienic article for personal wear by a male. The hygienic article includes an elongate, vapor permeable or breathable, and at least uni-directionally stretchable fabric sleeve having a first end, a longitudinally opposite second end and a central passage extending longitudinally between the first and second ends of the sleeve, the first end of the sleeve being open to the central passage to permit the wearer's penis to extend through said open first end into the central passage of the sleeve, the second end of the sleeve being closed and encompassing an absorbent cap, which controls leakage of liquid at the tip of the user's penis.

### BRIEF DESCRIPTION OF THE DRAWINGS

FIG. 1 is a perspective view of one embodiment of a hygienic article for receiving a penis of a male wearer;

FIG. 2 is a cross-section view of FIG. 1 taken in the plane of line 2-2 of FIG. 1;

FIG. 3 is a cross-section view of an alternative embodiment of FIG. 1;

FIG. 4 is a cross-section view of another alternative embodiment of FIG. 1;

FIG. 5 is a perspective view of another alternative embodiment of a hygienic article for receiving a penis of a male wearer;

FIG. 6 is a cross-section view of the embodiment of FIG. 5 taken in the plane of line 6-6 of FIG. 5;

FIG. 7 is a cross-section view of an alternative embodiment of FIG. 5;

FIG. 8 is an alternative perspective view of FIG. 4 showing the article in a storage configuration.

### DETAILED DESCRIPTION

For the purposes of this description, it should be recognized that the male penis includes an elongated shaft portion (which starts at a penis base, originating at the lower abdomen/pelvic region) and ending at a tip portion through which urine exits the body (known also as the glans penis).

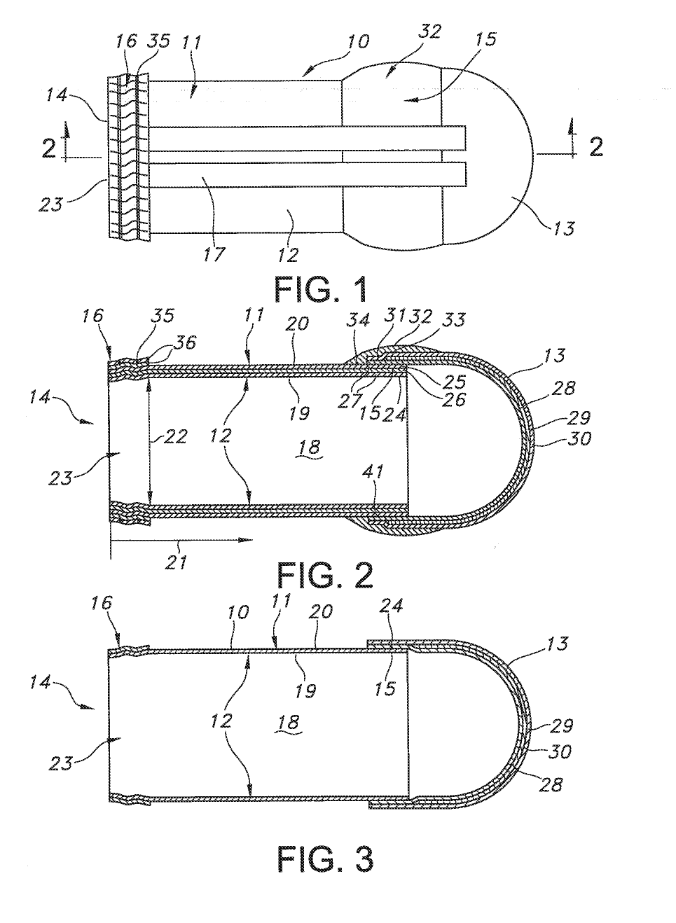

Referring now to the drawings and in particular to FIG. 1, one embodiment of a hygienic article 10 for personal wear by a male comprises in FIG. 1, an elongate uni-directionally stretchable or elastic fabric sleeve 11, or desirably a multidirectionally stretchable or elastic fabric sleeve 11, which is desirably breathable/vapor permeable and liquid permeable. The fabric sleeve 11 is suitably sized and shaped for receiving at least a portion of the length of a wearer's penis. The sleeve is comprised of a shaft portion 12 and a cap attachment portion at end 15. The shaft portion 12 is permanently affixed at one end (the cap attachment portion at end 15) to the cap 13, that is constructed to take-in and retain residual fluids (i.e., urine, semen, perspiration, etc.) commonly associated with the penis. The sleeve 11 includes an opening 23 at one end such as that shown in FIG. 1 for receiving the penis. The illustrated sleeve 11 has a first end 14 (in the shaft portion), a longitudinally opposite second end 15 (the cap attachment portion). As can be seen in FIG. 1, the article may optionally include an elastic collar 16 for providing additional securement of the article to the wearer during use, the collar to be positioned at or near the base of a user's penis shaft in use, and optional longitudinally directed adhesive patches 17 (shown with adhesive release sheets) along one surface of the sleeve 11, for additional securement of the article to a user's undergarments when in use. It should be recognized that the collar 16 may be placed at or near the first end 14 of the shaft portion. While not shown in the Figure, additional elastic collars may also be positioned along the sleeve 11 surface, such as are illustrated with respect to FIG. 5. While shown affixed to the exterior surface of the sleeve 11, collar 16 can just as easily be affixed to the interior surface of the sleeve or a separate collar can be affixed to both interior and exterior surfaces. Further, the optional elastic collar can be, in an alternative embodiment, incorporated within an internal portion of the product in the event the shaft portion is made from a laminate. Still further, the collar 16 may be located just inboard of the end 14 of the shaft portion and the shaft portion material closer to the end 14, may be folded over and cover the collar 16.

FIG. 2 illustrates a cross-section view of FIG. 1 taken along line 2-2. As can be seen in the Figure, a central passage 18 defined by a sleeve inner surface 19, extends longitudinally between the first and second ends (14, 15) of the sleeve. The sleeve also includes an outer surface 20.

The sleeve 11 has a longitudinal direction 21 extending longitudinally between the first 14 and second end 15 (second end with cap 13 attached) of the sleeve, and a transverse direction 22 extending across the sleeve. The first end 14 of the sleeve 11 is suitably open to the central passage of the sleeve to permit the wearer's penis to extend through the open first end into the central passage 18 of the sleeve with the first end of the sleeve positioned nearer to a base of the wearer's penis than the second end 15 of the sleeve. The shaft portion 12 of the sleeve 11 is permanently affixed at cap attachment portion/end 15 in attachment region 24 to a cap 13 for capturing and retaining fluid. Therefore the tubular structure of the article has one open end and one closed end.

The sleeve 11 may be sized for positioning of the first end 14 further away from the base of the penis so that the sleeve covers only a portion of the shaft of the penis, with the second end 15 of the sleeve still being disposed respectively inward of the tip of the wearer's penis. The fabric from which the sleeve 11 is constructed is desirably sufficiently flexible (stretchable or elastic in at least one direction), and vapor or liquid permeable, to permit for the natural daily movement of the penis, including contractions and expansions, without becoming dislodged, as well as for the passage of air and in some embodiments liquid. Therefore, in another embodiment, the sleeve fabric is desirably liquid permeable. While the article is designed to be constructed primarily from soft, uni or multi-directionally stretchable or elastic fabric, it may be desirable to include at least another elastic component or collar 16, for additional securement to the shaft of a penis. The collar 16 may be made from an elastic material such as a woven material, a foam, fibers, yarn or strands, film, nonwoven or a nonwoven laminate, or any combination thereof. An exemplary stretch bonded nonwoven laminate is described in U.S. Pat. No. 5,385,775 to Wright, hereby incorporated by reference in its entirety. In such a laminate, elastic strands 35 are bonded between two inelastic nonwoven sheets, such as spunbonded layers while such elastic strands are in an extended orientation. Upon removal of extension stress, such laminate contracts thereby forming a soft gathered or puckered fabric. Such fabric may demonstrate stretchable or elastic performance similar, or different from that of the underlying sleeve material. It would be important for such optional elastic collar to exhibit sufficient tension on the penis to retain such article in place without slipping, but not so much tension as to create red-marking or other sacrifice in comfort. Such collar 16 can be bonded to the underlying sleeve material at the outer 20 or inner surface 19, using traditional bonding techniques, such as by ultrasonic, adhesive, thermal or needle punch attachment. As noted, in use it may also be desirable to provide additional garment securement systems to the article, such that the article 10 does not move while being worn. In this regard, adhesive patches may be positioned at locations along the sleeve for adherence to a user's undergarments. If such adhesive attachment systems are deployed, the user would place the article upon his penis and then remove the release sheets while his penis is positioned in a downward direction facing his legs, such that the adhesive strips would face outward from his crotch region thereby easily adhering to the wearer's undergarments, roughly in the fly area, once the undergarments are positioned over the hygienic article. As shown in FIG. 1, the pressure sensitive adhesive patches may be multiple continuous longitudinal stripes 17. Alternatively they may be a single adhesive patch or a series of discontinuous adhesive patches in any shape or location which directly faces the wearer's undergarments.

The term “fabric” as in reference to the material from which the sleeve 11 is constructed, is intended to include foam, woven materials and knitted materials as well as to non-woven materials, which are fibrous webs or materials formed without the aid of a textile weaving or knitting process and laminates of such materials. Suitable materials from which the fabric may be formed include, without limitation, synthetic fibrous materials (for example, polyester polyethylene or polypropylene fibers), natural fibrous materials (for example, wood or cotton fibers), and combinations of natural and synthetic fibers. The fabric may be composed of single or multi-layered film materials as well, or alternatively laminates of fibrous materials and/or film materials. The fabric should be stretchable or elastic in at least a single direction, but desirably in multiple directions and fluid permeable, such that it allows both breathability and even more desirably for liquid permeation. The primary function of the sleeve portion of the absorbent article (in the shaft area) is not to retain fluid, but to allow for skin ventilation at least along the penis shaft. In further embodiments, where the sleeve 11 is a continuous inner article layer at the shaft 12 and cap 13 areas, it may also serve as a liquid permeable liner layer.

Suitable non-woven fibrous webs for use in constructing the sleeve 11 may be formed by any known technique for forming non-woven fibrous webs including, without limitation, carding, air forming, air laying, wet laying, co-forming and the like. Suitable fibers may be meltblown fibers, spunbond fibers or other suitably formed fibers. Meltblown fibers may be formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al. which is hereby incorporated by reference in its entirety. Meltblown fibers are microfibers which may be continuous or discontinuous, are generally smaller than about 0.6 denier, and are generally self bonding when deposited onto a collecting surface.

Spunbond fibers may be formed by extruding molten thermoplastic material as filaments from a plurality of fine capillaries of a spinnerette having a circular or other configuration, with the diameter of the extruded filaments then being rapidly reduced by a conventional process such as that described in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Peterson, and U.S. Pat. No. 3,542,615 to Dobo et al., each of which is incorporated herein in its entirety by reference. Spunbond fibers are generally continuous and often have average deniers larger than about 0.3, more particularly, between about 0.6 and about 10. As stated previously, laminates of such fibrous nonwoven webs, are also contemplated as base materials for such sleeve 11.

It is also contemplated that the fibers used in forming a suitable non-woven web for the sleeve 11 may be multi-component fibers (e.g., bicomponent fibers) in which two or more components are used in forming each fiber, such as in a sheath-core arrangement, side-by-side arrangement, island-in-a-sea arrangement or other suitable arrangement.

Other suitable fabrics, and more suitably non-woven materials, for use in making the sleeve 11 may be formed at least in part of water dispersible or flushable non-woven materials or a biodegradable non-woven materials such as aliphatic polyesters. The term biodegradable refers to the ability of a material to degrade due to the action of naturally occurring microorganisms such as bacteria, fungi, and algae. Various biodegradable aliphatic polyesters from which the non-woven web may be formed include, for example, polyesteramides, modified polyethylene terephthalate, polylactic acid (PLA), terpolymers based on polylactic acid, polyglycolic acid, polyalkylene carbonates (such as polyethylene carbonate), polyhydroxyalkanoates (PHA), polyhydroxybutyrates (PHB), polyhydroxyvalerates (PHV), polyhydroxybutyrate-hydroxyvalerate copolymers (PHBV), combinations thereof, and the like. Still other suitable fabrics for use in making the sleeve 11 include stretchable or elastic, and apertured polymeric films, or laminates of such films and previously described nonwoven materials.

In still a further alternative embodiment of the present invention, heat activatable/shrinkable and stretchable or elastic polymeric materials may be used which are activated by either normal body temperature heat or from external sources, such as blow dryers. In this fashion, a sleeve can be personally sized (based on heat shrinking) to conform to a desired shape, once applied to a consumer.

In one particularly suitable embodiment, the fabric sleeve may be an elastic and breathable/vapor permeable non-woven and film laminate fabric, such as that material described in US 2008/0132866 to Sigueira et al, which subject matter is hereby incorporated by reference hereto in its entirety. Such material is desirably formed of an apertured film and nonwoven bonded laminate, which includes an apertured film positioned between two soft nonwoven layers. The film may be made using either a cast or blown method. Desirably the film in such a laminate is formed from elastomers such as a propylene, polyethylene or styrenic block copolymer, and having a basis weight of between 15 gsm and 200 gsm, in another embodiment between about 30 and 45 gsm. The nonwoven layers are desirably formed from meltblown polyolefins or spunbonded polyolefins such as polypropylene, each having a basis weight of between 3 and 35 gsm, desirably between about 3 and 20 gsm. Desirably, the laminate including an apertured film and nonwoven components demonstrates an elasticity in at least one direction, such as for example the machine manufacturing direction of between about 50 and 300%, alternatively between 50 and 150%. Such material, also desirably demonstrates a breathability (air permeability) at least between 5 and 300 cfm, and in some embodiments, a liquid permeability as well. Such material can be further treated with surfactant, in order to enhance the level of liquid permeability. Desirably, such material demonstrates a high level of softness as well.

In order to determine values for material properties, the following test methods were utilized. For elastic performance, a cycle test was used, and for air permeability, a Frazier test was used.

### Cycle Testing

Material was tested using a cyclical testing procedure to determine load loss and percent set. In particular, 1-cycle testing was utilized to 150% defined elongation, but can also be used to higher levels of defined elongations. For this test, the sample size was 3 inches in the cross-machine direction by 6 inches in the machine direction. The grip size was 3 inches in width. The grip separation was 4 inches. The samples were loaded such that the machine direction of the sample was in the vertical direction. A preload of approximately 10 to 15 grams was set. The test pulled the sample to 100% elongation at a speed of 20 inches per minute, and then immediately (without pause) returned to the zero at a speed of 20 inches per minute. The results of the test data are all from the first cycle. The testing was done on a Sintech Corp. constant rate of extension tester 2/S with a Renew MTS mongoose box (control) using TESTWORKS 4.07b software (Sintech Corp, of Cary, N.C.). The tests were conducted under ambient conditions.

### Air Permeability

Air permeability was determined using Frazier permeability, which is measured as standard cubic feet per minute of air flow across a material, per square foot of material with an air pressure differential of 0.5 inches of water (125 Pa) across the sample. The test was performed at ambient conditions.

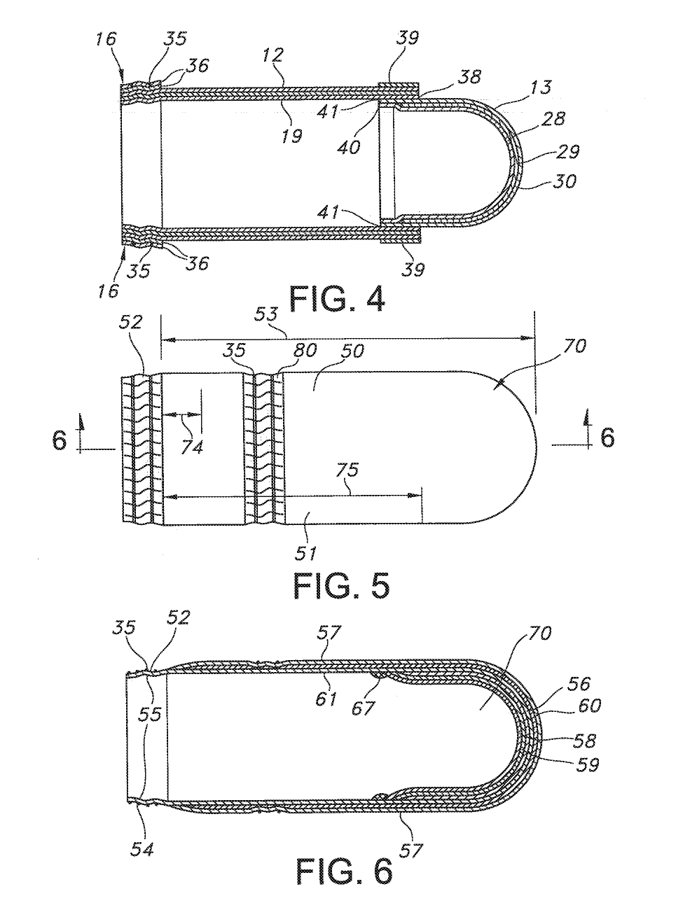

As can be seen in FIG. 2, an apertured film layer 26 may be bonded between two nonwoven layers 27 as described in the Sigueira noted above. In such a fashion, the interior and exterior surfaces, (19,20) of the sleeve 11 would be comprised of the soft nonwoven material. The sleeve material may either continue into the cap 13, at attachment region 24, or alternatively, be fastened along the outer surface of the cap 13, as can be seen in FIG. 4 at 38. The sleeve material may be bonded to the cap material using known bonding techniques such as adhesive, thermal, ultrasonic and needle punch bonding. It is particularly desirable to use such apertured film and nonwoven laminate material to entirely surround a sensitive body part, such as a penis, as such material demonstrates breathability/vapor permeability, elasticity, softness and desirably liquid permeability.

As can be seen in FIG. 2, the cap 13, desirably includes a liner layer 28 positioned adjacent to an absorbent layer 29. The absorbent layer 29 (which can be one or more layers that makes up the absorbent core of the product) is desirably sandwiched between the liner layer 28 and a liquid impermeable barrier layer 30. The liner layer 28 may be of a material traditionally used for liner or cover sheet applications in feminine, child care or adult care consumer product applications, such as for example a nonwoven or apertured film material used traditionally for feminine care pads or child care diapers. The absorbent layer, may be made of materials traditionally used for disposable absorbent consumer products such as thin feminine care liners or pads. Such materials may include in one embodiment, homogeneous composites of cellulosic materials or alternatively, mixtures of synthetic and cellulosic materials, such as superabsorbent polymers and cellulosic materials. The barrier layer, may be a single layer or multiple layers, such as a film and nonwoven laminate material, with the nonwoven layer desirably facing away from the first end 14. Such a material is also traditionally used in disposable consumer products as an outer cover material or baffle layer. Such a nonwoven outer layer would provide additional softness to the outside of the article. Desirably the liner and liquid impermeable barrier layers are bonded at their peripheries 31 such that their longitudinal and transverse dimensions are longer and wider than those of the sandwiched absorbent layer 29. Alternatively, a heavy seal through all layers to prevent wicking and leakage may be used at the end 15. Such layers may be bonded using traditional bonding techniques, such as adhesive, ultrasonic and thermal bonding methods. Alternatively, the liner, absorbent and barrier layers would each have the same longitudinal and transverse dimensions. Such layers can in one embodiment be bonded directly to the outside surface of the sleeve 20 at region 24, also by known bonding methods. Alternatively, an outer secondary bonding layer 32, such as a tape, film, or liquid impermeable nonwoven material may be used to bond/cover a portion of the cap 13 at attachment 33 and the sleeve 11 at attachment 34. Such outer bonding layer may be bonded to the sleeve and cap using the heretofore described bonding methods.

An article of the above construction would offer a user comfortable incontinence protection, that would be highly breathable in sensitive skin areas. Such an article would provide barrier protection at a targeted location near second end 15.

In another embodiment, as seen in FIG. 3, the fabric sleeve 11 is a single layer of any of the previously described breathable and/or liquid permeable materials that is bonded at the sleeve end 15 to the cap 13, again using any of the previously described bonding techniques. In another embodiment, as seen in FIG. 4, the shaft 12 is a laminate having an inner surface 19 bonded to the outer surface of a cap 13 at attachment region 38. The cap 13 has cap edges 41 positioned inside the shaft 12 along the inside surface 19. In particular, the inner surface 19 of the laminate would be bonded by traditional bonding techniques to the liquid impermeable barrier layer 30 of cap 13. As with the previous embodiment, a liner layer 28 and liquid impermeable barrier layer 30, would desirably sandwich the absorbent layer 29. In an alternative embodiment, a secondary elastic collar 39, could be positioned at the region of overlap between the second end 15 of the shaft portion and the cap edges 41. Such secondary elastic collar could be bonded using any of the heretofore described bonding techniques and could be constructed of materials described with respect to the optional elastic collar 16. Such secondary elastic collar may be used in connection with any of the embodiments described herein, so as to apply additional pressure to the region of the product in contact with the tip of a user's penis, so as to restrict the wicking of fluid contained in the cap 13 from traveling along the sleeve 11, thereby assisting in preventing leakage of liquid at the sleeve 11 area of the shaft 12.

In another embodiment of the article 50, having a length 53 excluding an elastic collar, as seen in FIG. 5, the sleeve 51 includes the unidirectional or multidirectional stretchable or elastic fabric previously described, but placed as the entire outermost layer of the article, such that a more streamlined outward impression is created for the product's appearance. In this fashion, the entire outer surface is comprised of the soft nonwoven layer of the unidirectional or multidirectional stretchable/elastic laminate, without a plastic-like exposed outermost surface, common to liquid impermeable film layers. No seams are visible between different materials on the outside of the product in this embodiment. As with the previous embodiment, an elastic collar 52 optionally with elastic strands 35 may be formed at or near one open end. An additional elastic collar 80, with optional elastic strands 35 may be placed between the open end of the article and a closed rounded end 70 where the cap is located. It should be recognized that the additional collar or collars can be placed in any of the embodiments included herein, and spaced periodically along the length. The elastic strands may be formed from elastic fibers or filaments. Alternatively, heat shrinkable or activatable polymer strands may be used.

The liner layer, sandwiched absorbent layer and barrier layers may be positioned either in a small overall portion of the length of the product towards the rounded end 70, leaving the majority of the product length 75 free from these cap materials, or alternatively along the majority of the inner product surfaces, leaving only a small portion of the product length 74 free from the cap materials.

As can be seen in FIG. 6, which shows a cross-section view of the article 50 of FIG. 5, taken along lines 6-6, the article includes an outer uni-directional or multi-directional laminate, itself constructed of three layers 57,60 and 61, desirably with nonwoven layers 57 and 61 sandwiching a film layer 60, as has previously been described. An internal absorbent cap at rounded end 70 includes a liner layer 58, absorbent layer 59, and liquid impermeable barrier layer 56 that are bonded to the inner surface of the laminate layer. Additionally, an optional hydrophobic barrier 67 is included at the junction of the nonwoven layer 61 and liner layer 58 to prevent wicking of liquid along the shaft of the article. Desirably the hydrophobic barrier 67 is a polymeric material, such as a hot-melt polymeric adhesive, which creates a wall-like structure that penetrates into the adjacent laminate layers 57, 60 and 61 and liner layer 58 to prevent wicking of fluid towards open end 55. Unlike the previous embodiments, in which the cap for capturing body fluids secreted by the penis is placed in a configuration extending away from the shaft end portion 15 of the sleeve 11, in the embodiment of FIG. 6 the absorbent cap is instead positioned inside the fabric sleeve which surrounds the absorbent components. The liner 58, absorbent layer 59, and liquid impermeable barrier layer 60 are bonded to the inner surface 61 of the shaft material of the surrounding sleeve, using any of the heretofore described bonding techniques. As with all embodiments described herein, such shaft and sleeve may be constructed of the materials described previously with respect to the sleeve/shaft, but desirably those described in the Sigueira reference.

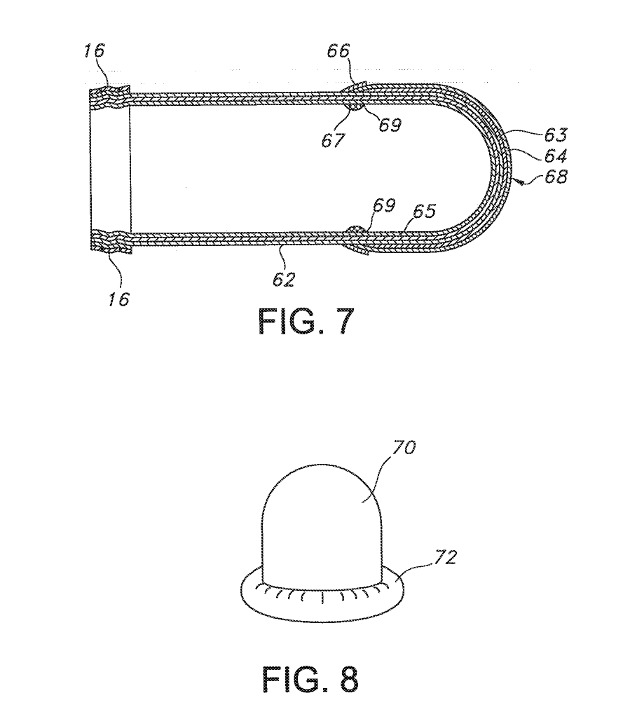

As can be seen in FIG. 7, which shows a cross-section view of an alternative embodiment of FIG. 4, instead of placement of an absorbent material on the inside of a sleeve 62 material, such liquid impermeable barrier layer 64 and absorbent layer 63 are bonded to the outside of the sleeve material 62 using any of the heretofore described bonding methods, and sandwiches the absorbent layer 63 between the sleeve material 62 (in particular a nonwoven layer of a three layer laminate as previously described) and the liquid impermeable barrier layer 64. In this embodiment, there is no separate liner layer in the construction, as the sleeve material itself 62 functions as the liner layer 65 immediately adjacent to the absorbent layer 63. In this embodiment, the apertured film and nonwoven laminate functions as a liner, as the apertures are sufficiently open to allow for the passage of liquid. So as to avoid the wicking of liquid along the sleeve shaft 62, an optional second elastic collar 66, or optional line of hydrophobic polymer 67, or both, may be placed at the overlap juncture of the sleeve material 62 with the absorbent cap 68 at region 69. Such collar can apply pressure to the region to form a seal, and such hydrophobic polymer can provide a structural barrier to prevent wicking, as with the prior embodiment. For enhanced liquid permeability in the cap 68, the sheath material 62 can be entirely or partially treated with a surfactant prior to manufacture.

As can be seen in FIG. 8, the article such as that shown in any of the previous figures, is desirably of sufficient flexibility to allow in one embodiment for the rolling of the sides into a storage configuration 70. The majority of the sleeve can be rolled outwardly 72, leaving only that portion of the article with the highest amount of absorbent layer per unit area in the cap unrolled. In this fashion, the article can be wrapped in an inconspicuous package such as those wrappers traditionally used to contain condoms. During use, a consumer need only unroll the article to the desired length after it is applied to the tip of a penis. If the article includes one or more elastic collars, as previously described in elements 16 and 52, such as in FIG. 5, the consumer need only unroll the article to the extent that reveals any of the elastic collars, for sizing or comfort. The article need not be entirely unrolled. Alternatively, the consumer may unroll the article completely on the penis. For removal of the article, the article may be pulled off as one would pull off a condom, or rolled back off.

As previously noted, the fabric sleeve 11 material is suitably stretchable, and more suitably elastic, in at least one of the transverse direction and longitudinal direction of the sleeve. Desirably, the fabric is multidirectionally stretchable or elastic. Allowing the sleeve to stretch in the transverse direction provides the sleeve with the ability to conform to the penis of the wearer and provide a snug, comfortable fit. Longitudinal stretchability allows the sleeve to conform to the length of the penis through various sizes (i.e., states of arousal, or natural expansion/contraction depending on environmental conditions, or daily activities) and positions of the penis. Accordingly, the sleeve is inhibited against inadvertent shedding from the wearer's penis during prolonged use. As used herein, the term “stretchable” refers to a material that may be extensible or elastomeric. That is, the material may be extended, deformed or the like, without breaking, and may or may not significantly retract after removal of an extending force. The term “elastic” refers to that property of a material where upon removal of an elongating force, the material is capable of recovering to substantially its unstretched size and shape or the material exhibits a significant retractive force. The term “extensible” refers to that property of a material where upon removal of an elongating force, the material experiences a substantially permanent deformation or the material does not exhibit a retractive force.

In one embodiment, elastic materials utilized in connection with the fabric sleeve desirably may be elongated/extended or stretched in at least one direction without breaking, by at least 25% (to at least 125% of its initial unstretched length) in at least one direction, suitably by at least 50% (to at least 150% of its initial unstretched length) and which will recover, upon release of the applied stretching or biasing force, at least 10% of their elongation. It is generally suitable that an elastic material or composite embodiment be capable of being elongated by at least 100%, more suitably by at least 200% or more, of its relaxed length and recover at least 30% and more suitably 50% of its elongation upon release of a stretching, biasing force, within about one minute.

Similarly, extensible or elongatable materials for use in making the sleeve 10 may in one embodiment be capable of stretching in at least one direction without breaking, by at least 25% (to at least 125% of its initial unstretched length) in at least one direction, suitably by at least 50% (to at least 150% of its initial unstretched length), more suitably by at least 100% (to at least 200% of its initial unstretched length). As an example, an extensible material having an initial unstretched length of 3 inches (7.6 centimeters) may be stretched without breaking to a stretched length of at least 3.75 inches (9.5 centimeters) in at least one direction (for the “by at least 25%” value).

In one particularly exemplary embodiment, the sleeve 11 may comprise a three ply laminate as described in US2008/0132866 wherein a propylene-based film is sandwiched between spunbonded nonwovens. Such a material is attached along its outside surface to the inner surface of an absorbent cap constructed of a 12 gsm polypropylene spunbond liner layer, a 150 gsm compressed homogeneous airlaid absorbent layer of cellulosic fibers, binder fibers and 20 percent superabsorbent materials, and a 20 gsm non-breathable polyethylene film/liquid impermeable barrier layer. The film barrier layer includes a laminated spunbond layer that faces away from the sleeve opening. A film-based adhesive tape holds the cap to the sleeve. An example of such an elastic sheath 11 may be as follows.

An elastic film was formed from 93 wt. % of propylene-based VISTAMAXX 1100 (Exxon Mobil Chemical Co.); 4 wt. % of Dow Polyethylene 6401 (Dow Chemical Co.); and 3 wt. % of a SCC116921 pigment (Standridge Color Corp.). The SCC116921 pigment contained titanium dioxide blended with polypropylene and polypropylene random copolymers. The polymers were compounded by weighing appropriate portions of pellets of each polymer, combining them into one container, and mixing them together by stirring. After compounding, the film samples were blown between a collapsing nip (operated at 38 feet per minute) so that a melt temperature of about 390° F. was achieved. The blown film was then thermally bonded between two polypropylene spunbond facings having a basis weight of approximately about 14 grams per square meter. Specifically, the film and facings were fed between an anvil and patterned roll (rib-knit). The patterned roll was heated to a roll surface temperature of 193° F., the anvil roll was heated to a roll surface temperature of 203° F., and a pressure of 117 pounds per linear inch. The rolls were operated at a speed of 210 feet per minute so that the film was stretched in the machine direction at a stretch ratio of about 5.5 (i.e., 5.5 times its original length). Once formed, the composite was then introduced into a nip of intermeshing grooved steel rolls, to stretch the composite in the cross machine direction. The grooved steel rolls were heated to a temperature of 125° F. Finally, the composite was then transferred to a winder, which operates at a speed of 79 feet per minute to allow the composite to retract. The final basis weight was approximately 98 grams per square meter.

As an additional feature, the sleeve 11 may have a suitable lubricious feature on its inner surface, such as by coating the inner surface with a lubricious element, by coating the individual fibers of the fabric sleeve with a lubricant, by impregnating the sleeve with such a lubricant or by other suitable applications. The lubricant is intended to facilitate sliding of the sleeve, first end first, onto the wearer's penis without causing damage or irritation thereto. Examples of suitable lubricants include, without limitation, silicones, cyclomethicones, dimethicones, dimethiconol, PEG dimethicone, alkyl silicones, phenyl silicones, silicone phospholipids, silicone gums, silicone oils, silicone waxes, cyclopentasiloxane, dimethicone crosspolymers, and combinations thereof. Particular lubricants suitable for use on the sleeve 10 include, for example, Dow Corning® 9041 Silicone Elastomer Blend Dimethicone (and) Crosspolymer, Dow Corning® 9011 Silicone Elastomer Blend, Dow Corning® 9040 Silicone Elastomer Blend—Cyclomethicone (and) Dimethicone Crosspolymer, Dow Corning® 9045 Silicone Elastomer Blend Cyclopentasiloxane (and) Dimethicone Crosspolymer, Dow Corning® 9509 Silicone Elastomer Suspension Dimethicone/Vinyl Dimethicone Crosspolymer (and) C12-14 Pareth-12, Dow Corning® 2503 Cosmetic Wax (Stearyl Dimethicone) Alkyl Methyl Siloxanes, Dow Corning® 556 Cosmetic Grade Fluid (polyphenylmethylsiloxane), Dow Corning® 929 Cat ionic Emulsion Amodimethicone (and) Tallowtrimonium Chloride (and) Nonoxynol-10, Dow Corning® HMW 222 0 Non-ionic Emulsion Divinyldimethicone/Dimethicone Copolymer (and) C12-C13 Pareth-3 (and) C12-C13 Pareth-23, Dow Corning® 2-8177 Emulsion Amodimethicone (and) C12-C14 Sec-Pareth-7 (and) C12-C14 Sec-Pareth-5, all of which are available from Dow Corning of Midland, Mich., U.S.A. It is understood that other types of lubricants could also be used to lower the dynamic coefficients of friction of the inner surface of the sleeve with respect to the skin of the wearer. It is also envisioned that the sleeve or at least the portion of the sleeve, such as the inside surface, can be made of a low friction material. In that case, the sleeve material would be considered the “lubricious feature”. The lubricant may also include a bacteriostatic, antifungal, or other suitable genito-urinary compatible treatment. Alternatively, a composition utilizing a drying agent such as talcum powder may be used in conjunction with application of the article.

In those embodiments described above, in which a separate liner 28, 58 is present in addition to the sleeve material, the liner 28 is generally designed to contact the body of the user and is liquid-permeable. The liquid permeable liner layer 28 has an outwardly facing surface that may contact the body of the wearer and receive aqueous fluids from the body. The liner is provided for comfort and conformability and functions to direct bodily exudates away from the body, through the liner and toward the absorbent layer 29,59. The liner retains little or no liquid in its structure so that it provides a relatively comfortable and non-irritating surface next to the tissues of the glans penis. The liner can be constructed of any woven or nonwoven material that is easily penetrated by bodily exudates contacting the surface. Examples of suitable materials include rayon, bonded carded webs of polyester, polypropylene, polyethylene, nylon, or other heat-bondable fibers, polyolefins, such as copolymers of polypropylene and polyethylene, linear low-density polyethylene, and aliphatic esters such as polylactic acid. Finely perforated film webs, spunbonded and net material can also be used. A specific example of a suitable liner material is a bonded carded web made of polypropylene and polyethylene such as that used as topsheet stock for KOTEX® pantiliners and obtainable from Sandler Corporation (Germany). U.S. Pat. No. 4,801,494 to Datta, et al. and U.S. Pat. No. 4,908,026 to Sukiennik, et al. teach various other topsheet materials that may be used in the present invention. Desirably, if present, the separate liner layer is present in a basis weight of between about 8 and 24 gsm. Alternatively, such separate liner layer may be present in a basis weight of between about 10 and 18 gsm. In a further alternative embodiment, such liner layer may be present in a basis weight of between about 10 and 12 gsm.

The liquid impermeable barrier layer 30,60,64 may permit a passage of air or vapor out of the article, while still blocking the passage of liquids, or may alternatively be non-breathable. Any liquid impermeable material may generally be utilized to form this barrier layer. For example, one suitable material that may be utilized is a microporous polymeric film, such as polyethylene or polypropylene. In particular embodiments, a polyethylene film is utilized that has a thickness in the range of about 0.2 mils to about 5.0 mils, and particularly between about 0.5 to about 3.0 mils. A specific example of a liquid impermeable material is a polyethylene film such as that used in KOTEX® pantiliners and obtainable from Pliant Corporation, Schaumburg, Ill., USA. Additional suitable liquid impermeable materials include multilayered films, liquid impermeable nonwoven layers and laminates thereof. In one embodiment, such a liquid impermeable barrier layer would have a basis weight of between about 16 and 40 gsm. In another alternative embodiment, such barrier layer would have a basis weight of between about 18 and 30 gsm. In still a further alternative embodiment, such barrier layer would have a basis weight of between about 10 and 24 gsm.

As indicated above, an absorbent layer 29,59,63 is positioned between the liner and the liquid impermeable barrier, and provides capacity to absorb and retain secretions from a user's penis. The absorbent core may be formed from a variety of different materials and contain any number of desired layers. For example, the core typically includes one or more layers of an absorbent web material of cellulosic fibers (e.g., wood pulp fibers), other natural fibers, synthetic fibers, woven or nonwoven sheets, scrim netting or other stabilizing structures, superabsorbent material, binder materials, surfactants, selected hydrophobic and hydrophilic materials, pigments, lotions, odor control agents or the like, as well as combinations thereof. In a particular embodiment, the absorbent web material includes a matrix of cellulosic fluff, and may also include superabsorbent material, such as for example in an airlaid material. The cellulosic fluff may comprise a blend of wood pulp fluff. One preferred type of fluff is identified with the trade designation NB 416, available from Weyerhaeuser Corp., and is a bleached, highly absorbent wood pulp containing primarily soft wood fibers. The absorbent materials may be formed into a web structure by employing various conventional methods and techniques. For example, the absorbent web may be formed with a dry-forming technique, an air forming technique, a wet-forming technique, or the like, as well as combinations thereof. A coform nonwoven material may also be employed, as may be a foam material. Methods and apparatus for carrying out such techniques are well known in the art. Given that the described article is concerned with providing relief from light incontinence, the absorbent layer is desirably made from a mixture of cellulosic fluff and superabsorbent polymer, or alternatively a compressed layer of an airlaid material. Such absorbent layer is desirably present in a basis weight of between about 100 to 400 gsm with between about 10 to 50 percent superabsorbent materials for odor control. In an alternative embodiment, the absorbent layer is desirably present in a basis weight of between about 120 to 250 gsm with between about 15 to 35 percent superabsorbent materials. In still a further alternative embodiment, the absorbent layer is desirably present in a basis weight of between about 140 and 160 gsm and with between about 20 to 25 percent superabsorbent materials.

The liner layer, where separate liners are present, may be maintained in secured relation with the absorbent layer (or liquid impermeable barrier layer) by bonding all or a portion of the adjacent surfaces to one another. A variety of bonding mechanisms known to one of skill in the art may be utilized to achieve any such secured relation. Examples of such mechanisms include, but are not limited to, the application of adhesives in a variety of patterns between the two adjoining surfaces, entangling at least portions of the adjacent surface of the absorbent layer with portions of the adjacent surface of the liner layer, or fusing at least portions of the adjacent surface of the liner layer to portions of the adjacent surface of the absorbent layer. The liner layer typically extends over the surface of the absorbent layer facing the penis, but can alternatively extend around the absorbent layer to partially or entirely, surround or enclose the absorbent layer. Alternatively, the liner layer and the liquid impermeable barrier layer can have peripheral margins that extend outwardly beyond the terminal, peripheral edges of the absorbent layer as illustrated, such as in FIGS. 2 and 3, and the extending margins can be joined together to partially or entirely, surround or enclose the absorbent layer (or multiple absorbent layers).

Although not required, the article may also contain other additional layers as is known in the art. For example, although not shown, a liquid-permeable intake layer (or surge layer) may be positioned vertically between the liner layer and the absorbent layer. The intake layer may be made of a material that is capable of rapidly transferring, in the z-direction, body fluid that is delivered to the liner layer. The intake layer may generally have any shape and/or size desired. The intake layer material may be synthetic, cellulosic, or a combination of synthetic and cellulosic materials. For example, airlaid cellulosic tissues may be suitable for use in the intake layer. The airlaid cellulosic tissue may have a basis weight ranging from about 10 grams per square meter (gsm) to about 300 gsm, and in some embodiments, between about 40 gsm to about 150 gsm. The airlaid tissue may be formed from hardwood and/or softwood fibers. The airlaid tissue has a fine pore structure and provides an excellent wicking capacity. The article may also contain a transfer delay layer/distribution layer (not shown) positioned between the intake layer and the absorbent layer. The transfer delay layer may contain a material that is substantially hydrophobic, such as a nonwoven web composed of polypropylene, polyethylene, polyester, etc. One example of a material suitable for the transfer delay layer is a spunbond web composed of polypropylene, multi-lobal fibers. Further examples of suitable transfer delay layer materials include spunbond webs composed of polypropylene fibers, which may be round, tri-lobal or poly-lobal in cross-sectional shape and which may be hollow or solid in structure. Typically the webs are bonded, such as by thermal bonding, over about 3% to about 30% of the web area. Other examples of suitable materials that may be used for the transfer delay layer 36 are described in U.S. Pat. No. 4,798,603 to Meyer, et al. and U.S. Pat. No. 5,248,309 to Serbiak, et al. To adjust performance, the transfer delay layer may also be treated with a selected amount of surfactant to increase its initial wettability. The transfer delay layer typically has a basis weight less than that of the other absorbent layers. The so-called distribution layer as is known in the art, may be present to evenly distribute liquid to the entire absorbent layer. Such a layer may be present in one embodiment between about 14 and 40 gsm, alternatively between about 16 and 30 gsm, and still further alternatively between about 18 and 20 gsm.

An optional impermeable bonding layer 32 of the above described embodiments is desirably formed of a liquid impervious polymeric film or nonwoven, bonded to the sleeve 11 and cap 13 with a suitable adhesive. The optional hydrophobic polymer 67 of the above described embodiments, is desirably formed in a continuous perimeter ring along the inside surface of the sleeve 11. The polymer desirably fills the interstitial spaces in underlying layers, thereby forming a dam for blocking the wicking of liquid from the cap 13 along the longitudinal direction of the sleeve 11. Such hydrophobic polymers are well known in the art. The article may be monochromatic for a user's discretion, or alternatively colored or ornamentally designed to be visually appealing to the consumer as well.

When introducing elements of the present invention or the preferred/desirably embodiments thereof, the articles “a”, “an”, “the”, and “said” are intended to mean that there are one or more of the elements. The terms “comprising”, “including”, and “having” are intended to be inclusive and mean that there may be additional elements other than the listed elements. As various other changes could be made in the above products without departing from the scope of the invention, it is intended that all matter contained in the above description and shown in the accompanying drawings shall be interpreted as illustrative and not in a limiting sense.

What is claimed is:

1. A hygienic article for personal wear by a male, said hygienic article comprising an elongate, liquid permeable, and at least uni-directionally stretchable fabric sleeve having a first end, a longitudinally opposite second end and a central passage extending longitudinally between the first and second ends of the sleeve, the first end of the sleeve being open to the central passage to permit the wearer's penis to extend through said open first end into the central passage of the sleeve, the second end of the sleeve being permanently affixed to an absorbent cap, which controls leakage of liquid at the tip of the user's penis.

2. The hygienic article of claim 1, wherein said stretchable sleeve portion is stretchable in multiple directions.

3. The hygienic article of claim 1, wherein said stretchable sleeve portion being elastic.

4. The hygienic article of claim 3 wherein said stretchable sleeve portion comprises a laminate of an apertured layer sandwiched between two nonwoven layers.

5. The hygienic article of claim 4, wherein the apertured layer is a film.

6. The hygienic article of claim 1, wherein said article further includes a means for preventing wicking of liquid along the sleeve of said article.

7. The hygienic article of claim 6, wherein said means includes at least one elastic collar.

8. The hygienic article of claim 6, wherein said means includes a hydrophobic polymer.

9. The hygienic article of claim 1, wherein said sleeve has an inside surface and an outside surface and said absorbent cap is attached to the outside surface of said sleeve.

10. The hygienic article of claim 1, wherein said sleeve has an inside surface and an outside surface and said absorbent cap is attached to the inside surface of said sleeve.

11. The hygienic article of claim 1 wherein said second end of said sleeve being closed, said closed end being bonded to an absorbent and liquid impermeable layer.

12. The hygienic article of claim 10, wherein said closed end of said sleeve serving as a liner layer adjacent an absorbent layer.

13. The hygienic article of claim 1, wherein said article is rolled upon itself along said sleeve for storage.

14. The hygienic article of claim 1, wherein said article includes at least one elastic collar along said sleeve.

15. The hygienic article of claim 14, wherein said article includes multiple elastic collars along said sleeve.

16. A hygienic article for personal wear by a male, said hygienic article comprising an elongate, liquid permeable, and at least uni-directionally stretchable fabric sleeve having a first end, a longitudinally opposite second end and a central passage extending longitudinally between the first and second ends of the sleeve, the first end of the sleeve being open to the central passage to permit the wearer's penis to extend through said open first end into the central passage of the sleeve, the second end of the sleeve being closed and encompassing an absorbent cap, which controls leakage of liquid at the tip of the user's penis.

17. The hygienic article of claim 16, wherein said stretchable sleeve being stretchable in multiple directions.

18. The hygienic article of claim 16, wherein said stretchable sleeve being elastic.

19. The hygienic article of claim 17 wherein said stretchable sleeve comprises a laminate of an apertured film sandwiched between two nonwoven layers.

20. The hygienic article of claim 16, wherein said article further includes a means for preventing wicking of liquid along the sleeve of said article.

21. The hygienic article of claim 20, wherein said means includes an elastic collar.

22. The hygienic article of claim 20, wherein said means includes a hydrophobic polymer.

23. The hygienic article of claim 16, wherein said sleeve has an inside surface and an outside surface and said absorbent cap is attached to the inside surface of said sleeve, said sleeve partially surrounding said cap.

24. The hygienic article of claim 16, wherein said article is rolled upon itself along said sleeve for storage.

25. A hygienic article for personal wear by a male, said hygienic article comprising an elongate, vapor permeable, and at least uni-directionally stretchable fabric sleeve having a first end, a longitudinally opposite second end and a central passage extending longitudinally between the first and second ends of the sleeve, the first end of the sleeve being open to the central passage to permit the wearer's penis to extend through said open first end into the central passage of the sleeve, the second end of the sleeve being closed and encompassing an absorbent cap, which controls leakage of liquid at the tip of the user's penis.
